SEQUENCE LISTING (15 sequences)

SEQ ID NO: 1 - DNA, 23 bp, Artificial Sequence
  PDGFRb target sequence
gcctggtcgtcacacccccgggg

SEQ ID NO: 2 - DNA, 22 bp, Artificial Sequence
  target sequence after treatment with Lacz-sgRNA
ggtcgtcacacccccggggcca

SEQ ID NO: 3 - DNA, 20 bp, Artificial Sequence
  target sequence after treatment with PDGFRBeta- sgRNA
ggtcgtcacacccggggcca

SEQ ID NO: 4 - DNA, 35 bp, Artificial Sequence
  target sequence after treatment with Lacz-sgRNA
cctggtcgtcacacccccggggccagagcttgtcc

SEQ ID NO: 5 - DNA, 33 bp, Artificial Sequence
  target sequences after treatment with PDGFRBeta-sgRNA
cctggtcgtcacacccggggccagagcttgtcc

SEQ ID NO: 6 - DNA, 34 bp, Artificial Sequence
  target sequences after treatment with PDGFRBeta-sgRNA
cctggtcgtcacaccccggggccagagcttgtcc

SEQ ID NO: 7 - DNA, 33 bp, Artificial Sequence; identical to SEQ ID NO: 5
  target sequences after treatment with PDGFRBeta-sgRNA

SEQ ID NO: 8 - DNA, 20 bp, Artificial Sequence
  PDGFRb target 1
acccgagcaggtcagaacga

SEQ ID NO: 9 - DNA, 20 bp, Artificial Sequence
  PDGFRb target 2
cgggttcagctccggtggtg

SEQ ID NO: 10 - DNA, 20 bp, Artificial Sequence
  PDGFRb target 3
gcctggtcgtcacacccccg

SEQ ID NO: 11 - DNA, 20 bp, Artificial Sequence
  PDGFRb target 4
gccccgggggtgtgacgacc

SEQ ID NO: 12 - DNA, 23 bp, Artificial Sequence
  Lacz sgRNA sequence
tgcgaatacgcccacgcgatggg

SEQ ID NO: 13 - DNA, 20 bp, Artificial Sequence
  sequencing primer
ggactatcatatgcttaccg

SEQ ID NO: 14 - DNA, 20 bp, Artificial Sequence
  PCR primer forward
ggcgagctgctgttgctgtc

SEQ ID NO: 15 - DNA, 20 bp, Artificial Sequence
  PCR primer reverse
aggtgccatcctgggccttg

(19) United States
(12) Patent Application Publication
(10) Pub. No.: US 2020/0179392 A1
(43) Pub. Date: Jun. 11, 2020

(54) Idelalisib for Treating Proliferative Vitreoretinopathy and Abnormal Intraocular Neovascularization

(71) Applicant: SCHEPENS EYE RESEARCH INSTITUTE, Boston, MA (US)

(72) Inventor: Hetian LEI, West Roxbury, MA (US)

(73) Assignee: Schepens Eye Research Institute, Boston, MA (US)

(21) Appl. No.: 16/320,790

(22) PCT Filed: Aug. 3, 2017
(86) PCT No.: PCT/US2017/045319

§ 371 Date: Jan. 25, 2019

Related U.S. Application Data
(60) Provisional application No. 62/371,396, filed on Aug. 5, 2016.

(51) Int. Cl. A61K 31/52; A61K 9/00; A61P 27/00

(52) U.S. Cl. CPC A61K 31/52; A61P 27/00; A61K 9/0019

(57) ABSTRACT

Methods using idelalisib to treat proliferative vitreoretinopathy and intraocular pathological angiogenesis (e.g., proliferative diabetic retinopathy (PDR), retinopathy of prematurity (ROP), and wet age-related macular degeneration (AMD)).

### CLAIM OF PRIORITY

This application claims the benefit of U.S. Provisional Patent Application Ser. No. 62/371,396, filed on Aug. 5, 2016. The entire contents of the foregoing are hereby incorporated by reference.

### FEDERALLY SPONSORED RESEARCH OR DEVELOPMENT

This invention was made with Government support under Grant No. RO1 EY012509 awarded by the National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on Aug. 3, 2017, is named 00633-0220WO1-ST25.txt and is 3,358 bytes in size.

### TECHNICAL FIELD

Described herein are methods using idelalisib to treat proliferative vitreoretinopathy (PVR) and intraocular pathological angiogenesis (e.g., proliferative diabetic retinopathy (PDR), retinopathy of prematurity (ROP), and wet age-related macular degeneration (AMD)).

### BACKGROUND

During the pathogenesis of PVR, retinal pigment epithelial (RPE) cells are exposed to vitreous during open optimal injury or retinal attachment surgery. These cells then migrate, manage to survive in the alien vitreous environment, proliferate, growth and secrete extracellular matrix, resulting in the formation of subretinal or epiretinal membranes whose contraction causes retinal detachment¹. PVR is a vision-threatening complication that develops in 8-10% of patients undergoing retinal attachment surgery after a primary retinal detachment²⁻⁷ and in 40-60% of patients with an open-globe injury^{8-16.} At present, repeat surgery is the only option to treat PVR, but this surgery has poor functional results. Although the efforts have been made to identify nonsurgical approaches to prevent PVR, they have not been successful.

Pathological angiogenesis is associated with PDR, ROP, and wet AMD.¹⁷ PDR accounts for the highest incidence of acquired blindness in the working-age population^{18,19}; ROP is a major cause of acquired blindness in children²⁰; and AMD represents the leading cause of blindness in people over the age of 65, afflicting 30-50 million people globally²¹. Without timely treatment, the new fragile vessels leak blood into vitreous, blur vision, destroy the retina and lead to blindness. Preventing vascular endothelial growth factor (VEGF)-stimulated activation of its receptors with neutralizing VEGF antibodies (ranibizumab & bevacizumab) and a recombinant fusion protein with the partial extracellular domains of VEGF receptor (VEGFR) 1 and 2 (aflibercept) has become an important therapeutic approach to treating abnormal angiogenesis in these eye diseases^{20, 21}. While anti-VEGF drugs can lessen vessel leakage and angiogenesis in many patients with these eye diseases, some patients are not responsive to these drugs²², so novel therapeutic approaches are required for such patients.

### SUMMARY

Phosphoinositide 3-kinases (PI3Ks) play a critical role in transmitting signals from cell surface molecules by phosphorylating the 3-hydroxyl of inositol membrane lipids. As demonstrated herein, among eye-originated cell lines PI3Kδ is highly expressed in retinal pigment epithelial (RPE) cells and human retinal microvascular endothelial cells (HREC). Idelalisib at its selective dose for PI3KO prevented vitreous- and VEGF- but not platelet-derived growth factor (PDGF)-induced activation of Akt. Moreover, idelalisib inhibited vitreous-stimulated proliferation, survival, migration and contraction of RPE cells, as well as PVR induced by RPE cells in a rabbit model. These results identify idelalisib as a novel therapeutic intervention in RPE-related pathologies such as PVR. In addition, idelalisib prevented VEGF-induced proliferation, migration and tube formation of HRECs.

Thus, provided herein are methods for treating or reducing the risk of intraocular diseases, e.g., PVR and intraocular pathological neovascularization, e.g., PDR, ROP, or wet AMD, in a subject. The methods comprise administering a therapeutically effective amount of idelalisib, e.g., locally to the eye, e.g., via intravitreal injection.

Also provided herein are compositions comprising idelalisib for use in treating or reducing the risk of intraocular diseases, e.g., PVR and intraocular pathological neovascularization, e.g., PDR, ROP, or wet AMD, in a subject. In some embodiments the compositions are formulated for local administration to the eye, e.g., by intravitreal or intraocular administration.

In some embodiments the subject is undergoing or has undergone an ocular surgical procedure that increases the subject's risk of developing PVR; has diabetes, e.g., diabetic retinopathy; is a pre-term infant born before 32 weeks' gestation; or has early stage AMD.

In some embodiments the ocular surgical procedure is a pars plana vitrectomy (PPV), retinal detachment (RD) surgery; epiretinal membrane (ERM) surgery; scleral buckle surgery; or a procedure in the other eye.

In some embodiments the subject requires a PPV to treat a rhegmatagenous retinal detachment secondary to trauma; preexisting PVR; or for other indications associated with high risk condition for PVR development.

In some embodiments the indication associated with high risk condition for PVR development is a giant retinal tear, a retinal break larger than 3 disc areas, a long-standing retinal detachment, or a detachment associated with hemorrhage.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-B. Idelalisib preferentially inhibited vitreous-induced activation of Akt in RPE cells

1A. The clarified lysates of HCF (human corneal fibroblast), RCF (rabbit conjuctival fibroblast), HTB-18 (human retinoblastoma), 661W mouse cone cells, PAEC (porcine aortic endothelial cells), hPRPE (human primary retinal epithelial cells), ARPE-19 (a human spontaneously arising RPE cell line), RPEM that are RPE cells from an epiretinal membrane of a patient with PVR, HREC (human retinal microvascular endothelial cells), HUVEC (human umbilical vascular endothelial cells), and HLEC (human lymphatic endothelial cells) were subjected to a western blot analysis using indicated antibodies. This represents three independent experiments.

1B. After growth factor-deprivation, the RPEM cells were pretreated with idelalisib at indicated concentrations, or its vehicle for 30 minutes, and then treated with normal rabbit vitreous diluted (1:2) in DMEM (RV), V: vehicle (0.1% dimethyl sulfoxide) or PDGF-B (10 ng/ml). After the treatment for 30 minutes at 37° C., their lysates were subjected to a western blot analysis using the indicated antibodies. This is representative of three independent experiments.

FIGS. 2A-B. Vitreous induced activation of PDGFRβ/PI3Kδ/Akt in RPE cells

2A. Serum-starved RPEM cells as denoted were treated with RV or PDGF-B for 30 minutes and their lysates were analyzed with western blot using indicated antibodies. This is a representative of three independent experiments. sgRNA: single guide RNA targeting lacZ (lacZ) or PDGFRB (PB3).

2B. Serum-deprived RPEM cells as defined were treated with RV or PDGF-B for 30 minutes and their lysates were analyzed with indicated antibodies. This is a representative of three independent experiments. βΔx: a truncated PDGFRβ without the PDGF-binding domain. NAC: N-acetyl-cysteine (10 mM), a scavenger of reactive oxygen species (ROS), SU: SU6656 (1 μM), a specific inhibitor of Src family kinases (SFKs).

FIGS. 3A-D. Idelalisib prevented vitreous-induced cellular responses

3A. RPEM cells were seeded into a 24-well plate at a density of 3×10⁴ cells/well. After 8 hours the cells had attached, the medium was changed to either 0.5 ml DMEM (−) or RV supplemented with idelalisib (5 μM) or its vehicle. After treated for 48 hours, the cells were counted with a hemocytometer under a light microscope.

3B. After RPEM cells reached 80% confluence in 60-mm dishes, the cultured medium was changed to either 3 ml DMEM/F12 or RV supplemented with idelalisib (5 μM) or its vehicle (0.1% dimethyl sulfoxide: DMSO). 48 hours later, the detached cells were washed with phosphate-buffer saline (PBS) and stained with fluorescein isothiocyanate (FITC)-conjugated Annexin V and propidium iodide (PI) and then analyzed by fluorescence-activated cell sorting (FACS). A representative raw data of three experiments below the bar graphs is shown.

3C. After the RPEM cells in a 12-well plate reached 90% confluence, the cultured wells were scratched with a 200 μl pipet tip and treated with RV supplemented with idelalisib (5 μM) or its vehicle. 16 hours later the pictures of the cells were taken and analyzed. A representative raw data of three experiments below the bar graphs is shown. Scale bar: 500 μm.

3D. The RPEM cells were re-suspended in 1.5 mg/ml of neutralized collagen I at a density of 1×10⁶ cells/ml and seeded into wells of a 24-well plate. The solidified gels were overlaid with either DMEM/F12 alone (−), or RV supplemented with idelalisib (5 μM) or its vehicle as indicated. After 48 hours the gel diameter was measured and the gel area were calculated. A photograph of a representative experiment is shown at the bottom of the bar graphs. The mean±standard deviation (SD) of three independent experiments is shown; * denotes power (p)<0.05 using an unpaired t-test.

FIGS. 4A-D. Idelalisib prevented experimental PVR in rabbits.

PVR was induced in the right eyes of two to three-month-old Dutch Belted pigmented rabbits. Briefly, one week after gas vitrectomy, rabbits were intravitreally injected with platelet-rich plasma (PRP, 0.1 ml) and RPEM cells (300,000 cells) supplemented with either idelalisib (10 μM) or its vehicle (0.1% DMSO). Subsequently, the rabbits were examined at the indicated times and PVR stage was evaluated using a double blind approach with an indirect ophthalmoscope through a 30D lens, and then the PVR status for each rabbit was plotted (4A). The power (P) values are indicated at the top of the figure after a Mann-Whitney analysis. Representative rabbits on day 28 were examined by eletroretinography (ERG) (4B), optical coherence tomography (OCT) (4C) and histological analysis by hematoxylin & eosin stain (4D). The numbers in C and D (0, 2, 3, and 5) denote PVR stages in the examined rabbits. Single arrow points to the retina and double arrows point to cellular fibrotic membranes (4C). Arrowhead and double arrows indicate cellular membranes (4D).

FIGS. 5A-B. Vitreous and PDGF induce activation of differential PI3Ks in hPRPE cells. Serum-starved hPRPE cells were pretreated with idelalisib (0.1, 1, 10, 20 μM) (A), a specific inhibitor of PI3Kδ, or GSK2636771, a specific inhibitor of PI3Kβ, (0.1, 1, 10, 20 μM) (B) for 30 minutes, and then treated with normal rabbit vitreous (RV, 1:2 dilution in DMEM) or PDGF-B (long/ml) for additional 30 minutes. Their clarified lysates were subjected to a western blot analysis using indicated antibodies. V: vehicle (0.1% DMSO). This is representative of three independent experiments.

FIGS. 6A-E. Depletion of PDGFRβ using CRISPR/Cas9. The four lentivirues generated by lentiCRISPR v2 vector with four different sgRNAs were used to infect ARPE-19 cells. The lysates from the puromycin-resistant cells were subjected to a western blot analysis with indicated antibodies (A). The PB3 and lacZ viruses were used to infect RPEM cells, respectively. The DNA fragment around the protospacer adjacent motif (PAM) sequence from the genomic DNA of the puromycin-selected cells were amplified by PCR and the PCR products were subjected to a nuclease surveyor assay (B), Sanger DNA sequencing (C) and next generation sequencing (D). The sequences shown in FIG. 6C are as follows: target sequence: GCCTGGTCGTCACACCCCCGGGG, SEQ ID NO:1; target sequence after treatment with LaczsgRNA: GGTCGTCACACCCCCGGGGCCA, SEQ ID NO:2; target sequence after treatment with PDGFR/3sgRNA GGTCGTCACACCCGGGGCCA, SEQ ID NO:3. The sequences shown in FIG. 6D are as follows: target sequence after treatment with LaczsgRNA CCTGGTCGTCACACCCCCGGGGCCAGAGCTTGTCC, SEQ ID NO:4; target sequences after treatment with PDGFRβ-sgRNA: CCTGGTCGTCACACCCCCGGGGCCAGAGCTTGTCC, SEQ ID NO:4; CCTGGTCGTCACACCC--GGGGCCAGAGCTTGTCC, SEQ ID NO:5; CCTGGTCGTCACACCCC-GGGGCCAGAGCTTGTCC, SEQ ID NO:6; and CCTGGTCGTCACACCC---GGGGCCAGAGCTTGTCC, SEQ ID NO:7. The lysates from the selected cells were applied to a western blot analysis using indicated antibodies (E). Arrows point an expected cleavage site by SpCas9, a star points to a nucleotide deletion, short underlines indicate PAM sequences, and short dashes indicate deleted nucleotides. These are representative of two independent experiments.

FIG. 7. Toxicity test of idelalisib in cultured cells. After RPEM^{23, 24} cells reached 90% confluence in a 24-well plate, they were treated with a serially increasing concentration of idelalisib (0, 2.5, 5, 10, 20 and 40 μM) in a serum-free DMEM/F12 medium, respectively²⁵. The pictures shown were taken on the RPEM cells treated for 48 hours. Scale bar: 200 μm. This is representative of two independent experiments.

FIGS. 8A-C. Examination of idelalisib toxicity in rabbit eyes²⁴. Idelalisib was injected into the rabbit vitreous to achieve a final vitreal concentration of 10 or 20 μM. This day was considered day 0. The rabbits underwent fundus examinations on days 1, 3, 5, and 7. On day 7, the rabbit eyes were examined by ERG (dark adaption) (A) and OCT (B). Subsequently, the eyeballs from the euthanized rabbits were subjected to histological analysis by hematoxylin & eosin stain (C). Arrows in B point to retinas. Representative data are presented in each panel for the indicated concentration. Scale bar: 200 μm.

FIG. 9. Diagram illustrating an exemplary pathway by which idelalisib, a specific inhibitor for PI3Kδ, may inhibit the vitreous/VEGF-stimulated activation of PI3Kδ/Akt signaling pathway and cellular events (e.g., proliferation, survival, and migration), and the pathology (e.g. PVR and angiogenesis). VEGF: Vascular endothelial growth factor, PI3Kδ: Phosphoinositide 3-kinase δ; RPE: retinal pigment epithelial cells; ECs: endothelial cells; PVR: proliferative vitreoretinopathy.

FIGS. 10A-C. Idelalisib inhibits VEGF-induced cell proliferation, migration and tube formation. A, Cell proliferation assay. B, Wound healing assay. C, Tube formation assay. Each bar graph indicates mean±standard deviation of three independent experiments. “*” denotes significant difference between the two compared groups. P<0.05 using an unpaired t-test.

FIGS. 11A-B. Idelalisib inhibits angiogenesis in a mouse model of oxygen-induced retinopathy (OIR). Litters of P12 mice that had been exposed to 75% oxygen for five days were injected intravitreally with 1 μl ilidelalisib (50 μM) or its vehicle (DMSO). A, On P17, whole-mount-retinas were stained with IB4. B, Analysis of neovascularization (NV) areas from the IB4 stained retinas (n=6). The bar graph data are mean±SD of three retinas. “*” indicates significant difference using an unpaired t test. p<0.05.

### DETAILED DESCRIPTION

As noted above, alternatives to surgical intervention for PVR and anti-VEGF for other diseases associated with intraocular pathological angiogenesis (e.g. PDR, ROP and wet AMD) are needed.

Phosphoinositide (PI) 3-kinases (PI3Ks), a family of lipid kinases, play an important role in transmitting signals from cell surface molecules such as receptor tyrosine kinases^{25, 26} and they are divided into three classes: I, II and III^{26, 27}. Idelalisib is a selective inhibitor for PI3Kδ, one of the PI3K class I. Upon appropriate stimulation, p85, a regulator subunit of PI3Kδ, can bind to the phosphorylated tyrosine at receptor tyrosine kinases so that the catalytic subunit (p110δ) can phosphorylate PI(4,5)P2 to become PI(3, 4, 5)P3, which in turn can be bound by Akt, an oncogene product also known as protein kinase B²⁸⁻³¹. This binding facilitates Akt to be phosphorylated by PI-dependent kinase I at threonine 308 and by the mammalian target of rapamycin complex 2 at serine 473³². Activation of Akt can stimulate multiple cellular processes such as cell survival, proliferation, and migration^{32, 33} Thus PI3Ks play an essential role in transmitting signals from cell surface molecules into the intracellular enzymes and in stimulating cellular responses^{25, 26, 31}. Deregulation of PI3Ks/Akt signaling pathway may initiate multiple diseases including pathological angiogenesis³⁴⁻³⁹ and proliferative vitreoretinopathy (PVR)⁴⁰⁻⁴³.

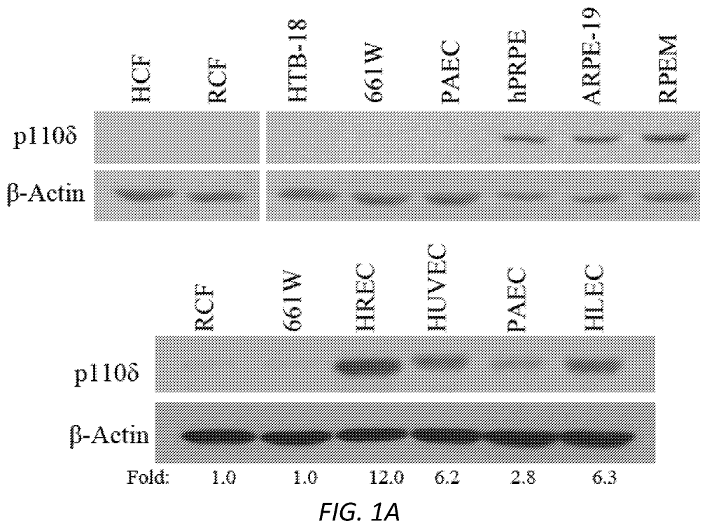

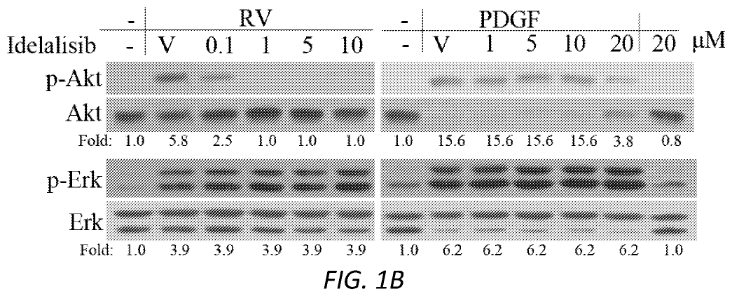

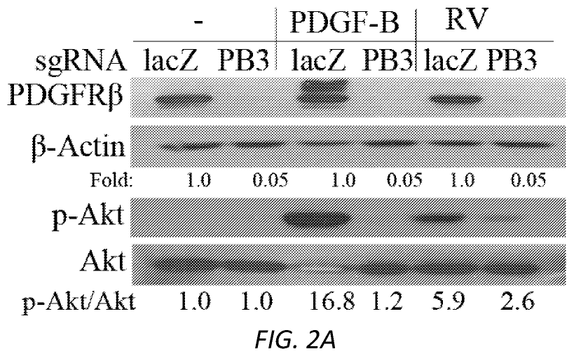

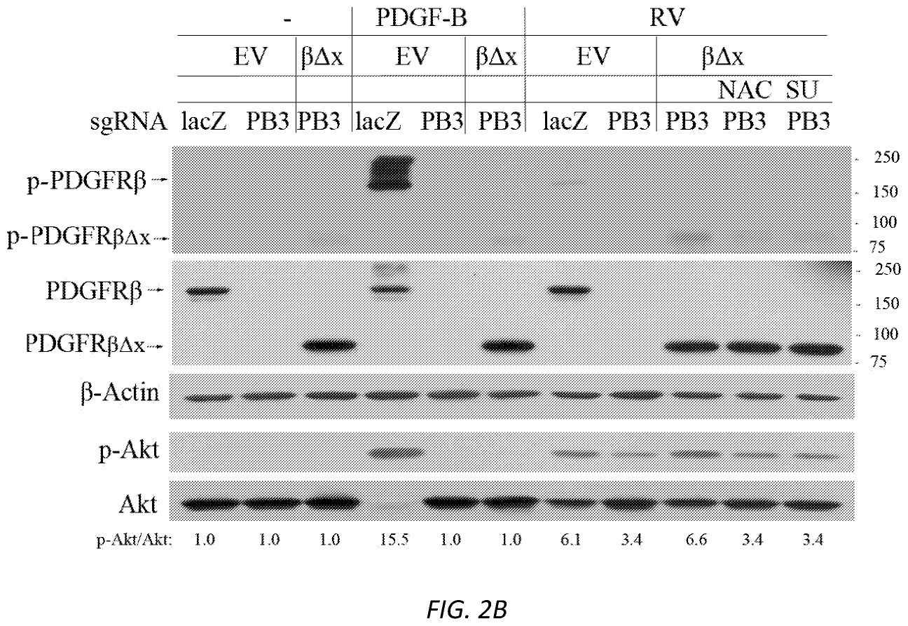

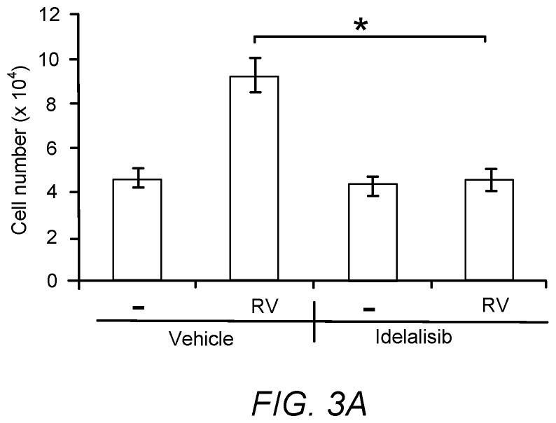

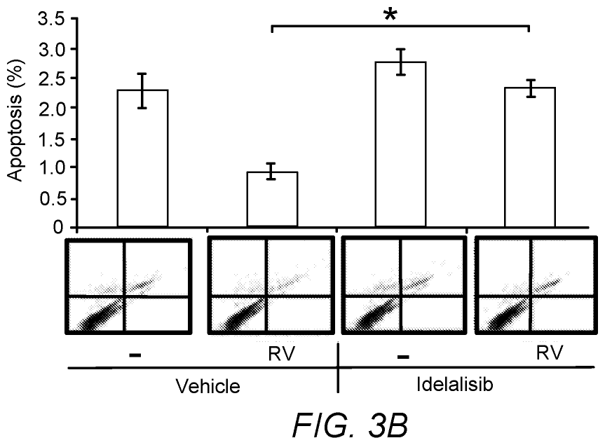

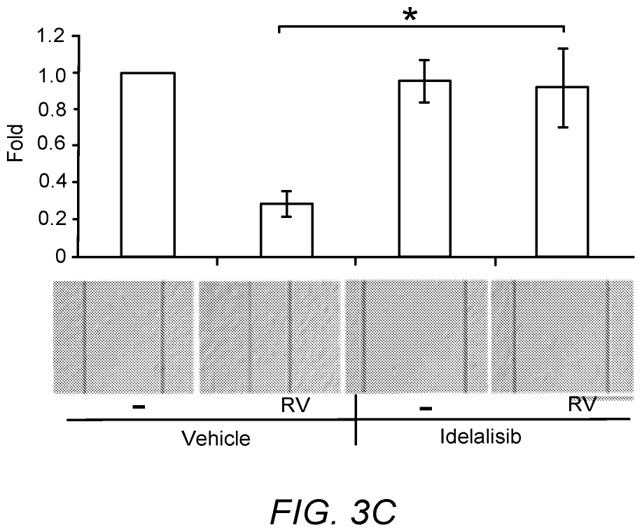

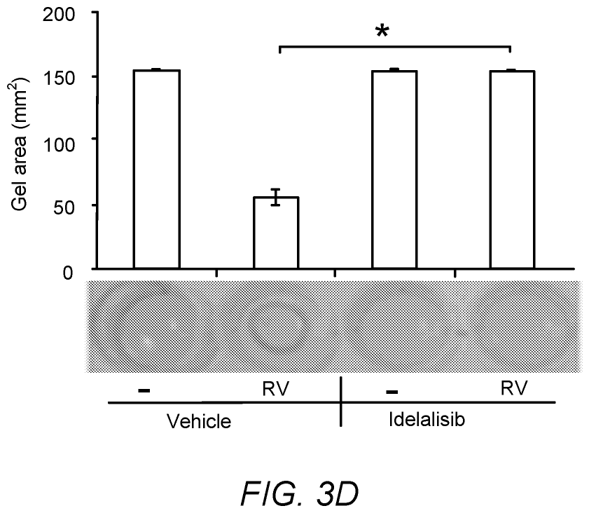

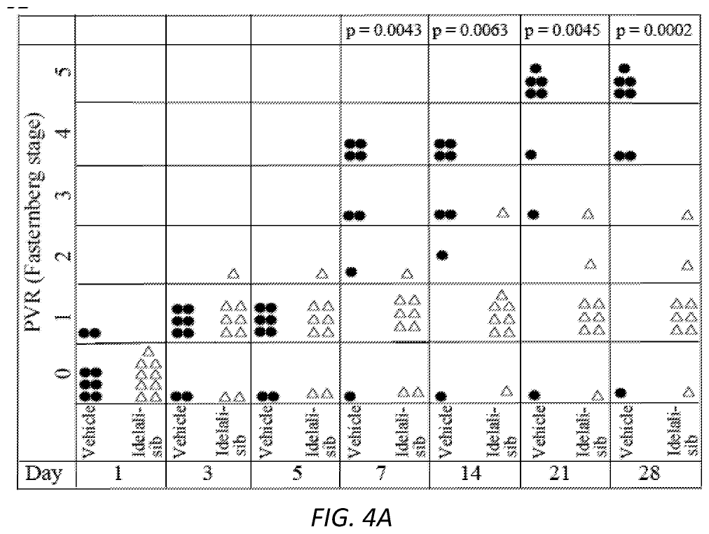

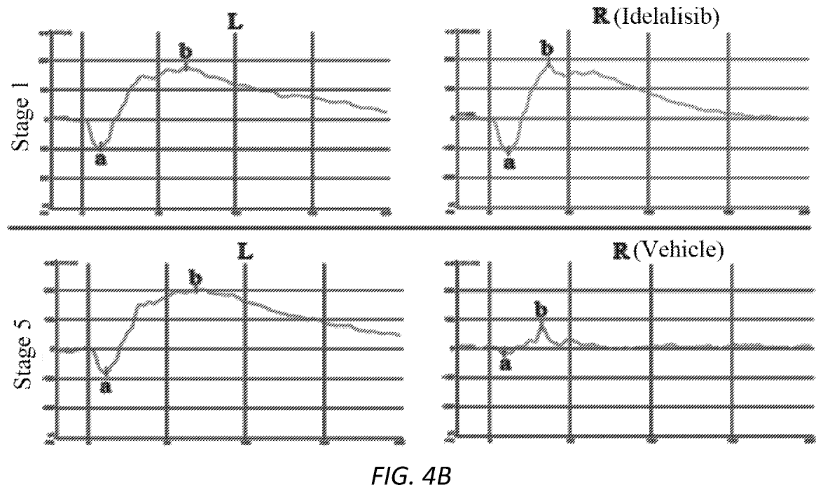

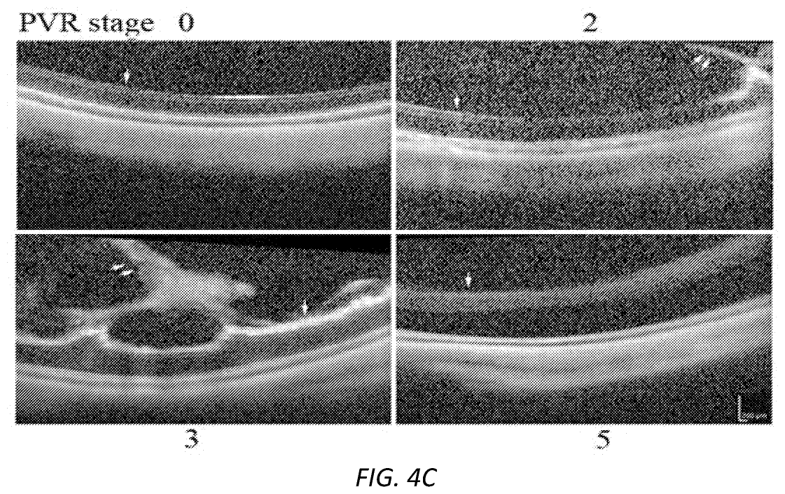

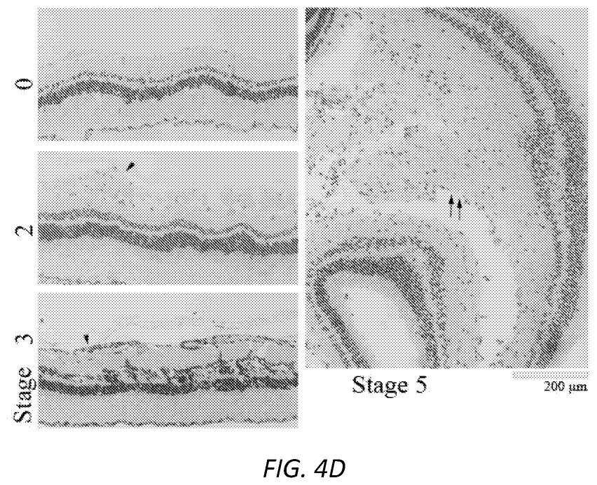

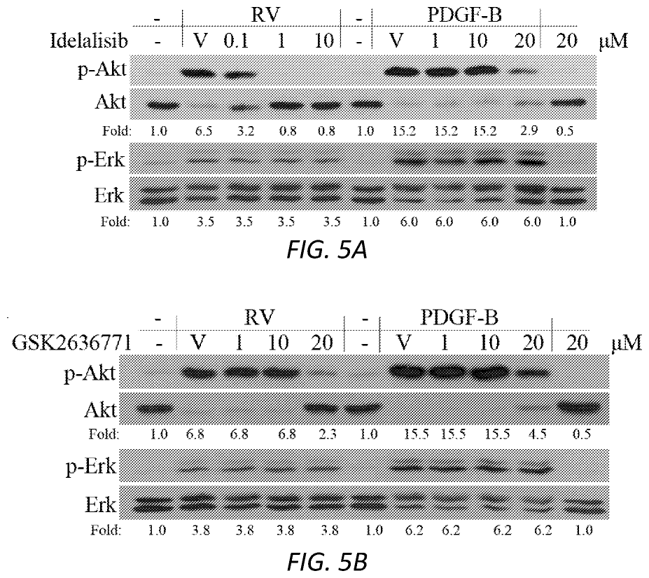

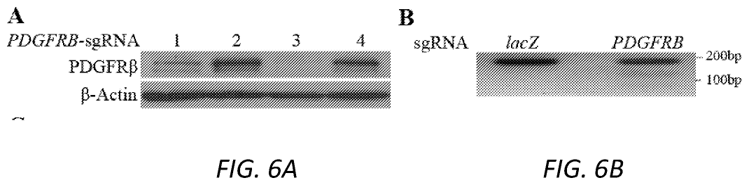

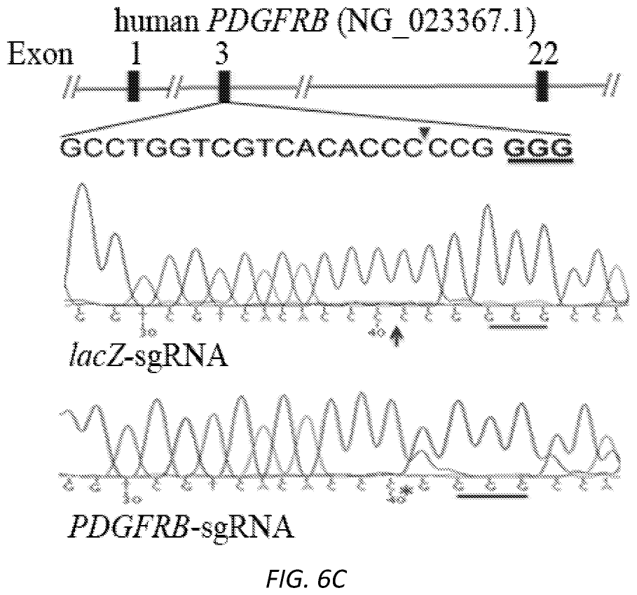

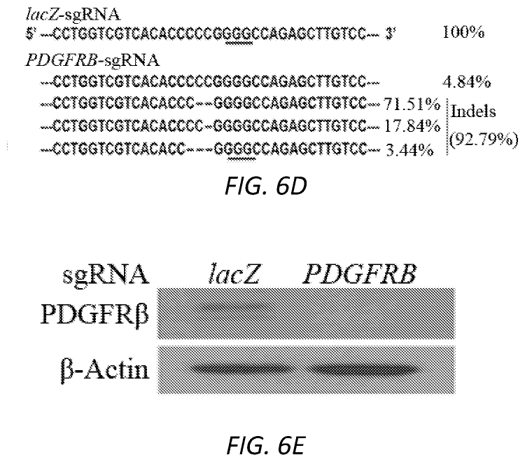

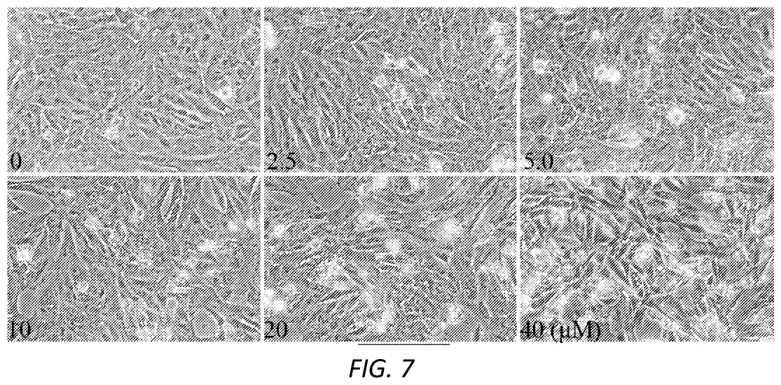

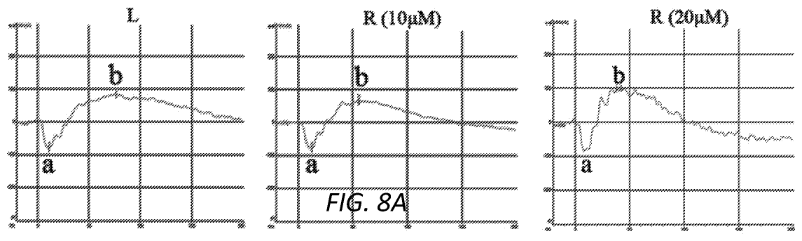

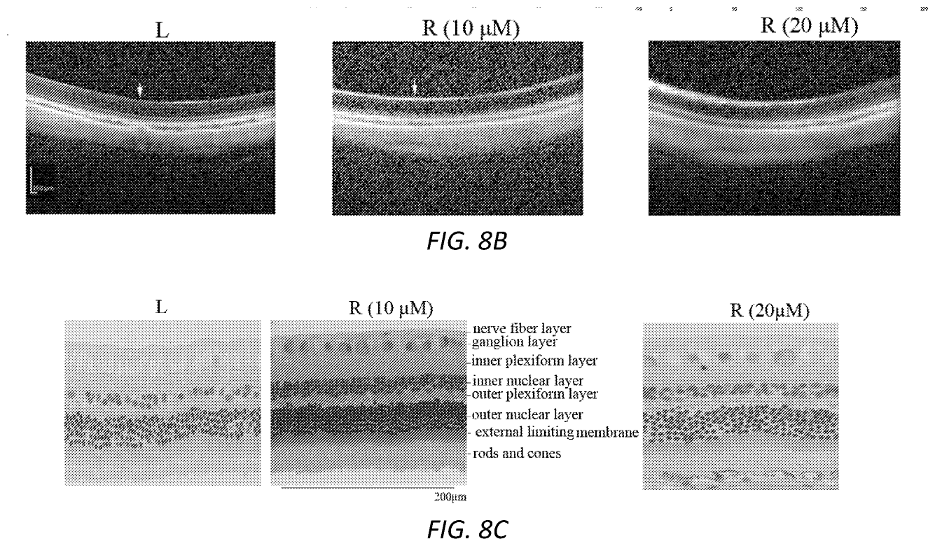

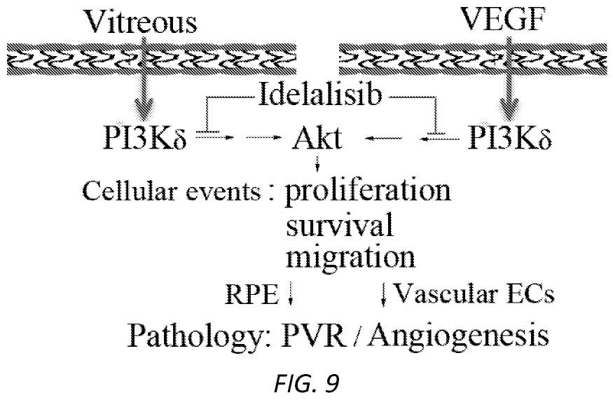

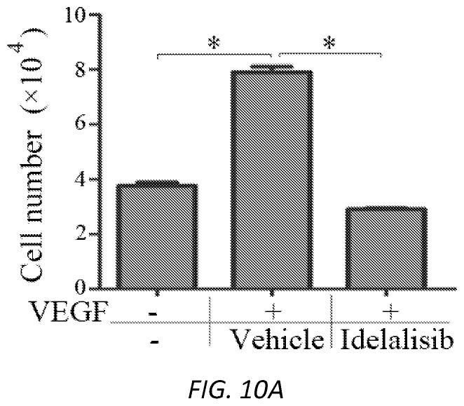

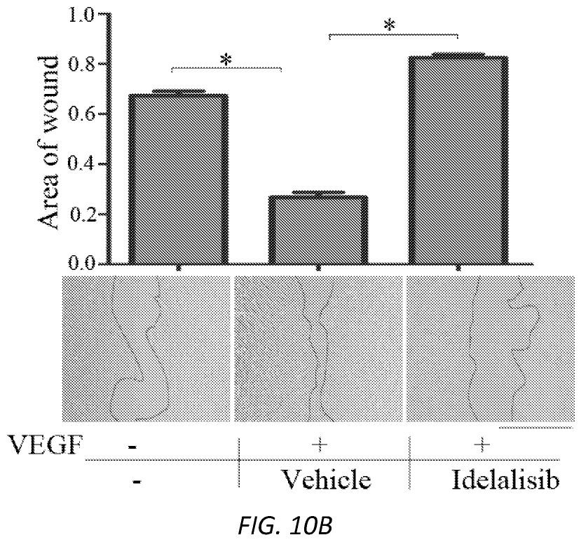

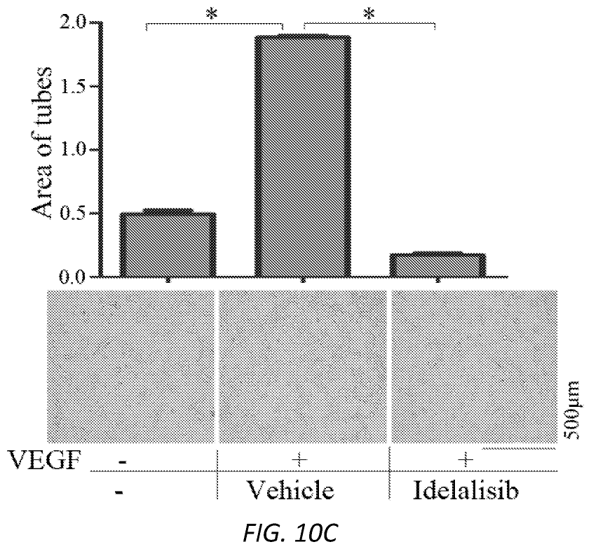

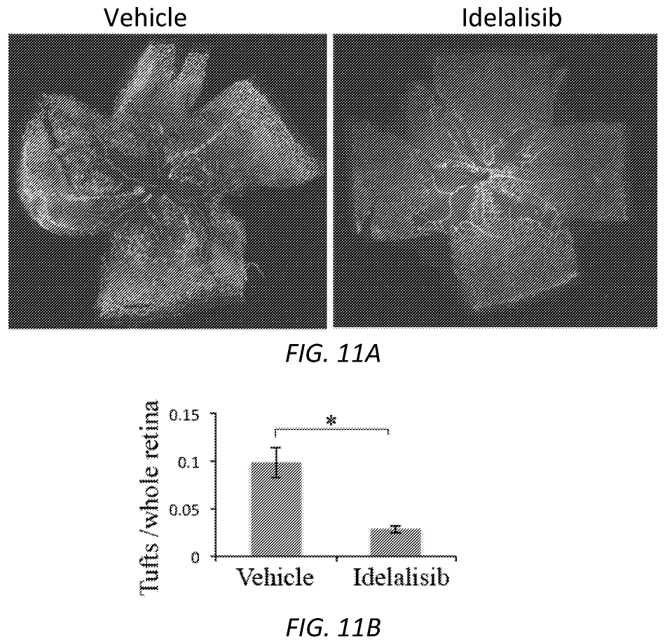

As described herein, human RPE cells and human retinal microvascular endothelial cells (HRECs) highly express p110δ (FIG. 1A). Idelalisib, a selective inhibitor for PI3Kδ approved by United States Food and Drug Administration for treating chronic lymphocytic leukemia, preferentially inhibited vitreous- and VEGF-induced activation of Akt (FIG. 1B), proliferation, survival and migration of RPE cell (FIGS. 3A-D) as well as HRECs (FIG. 10). In addition, we have found that idelalisib inhibited PVR induced by RPE cells in a rabbit model (FIGS. 4A-D and FIGS. 8A-C) and that idelalisib prevented VEGF-induced tube formation, an in vitro angiogenesis model (FIGS. 10A-C), and neovascularization in vivo (FIGS. 11A-B).

In summary, the present inventors have discovered that idelalisib, a specific inhibitor of PI3Kδ, which as shown herein is highly expressed in human RPE cells and HRECs, preferentially blocked vitreous-induced activation of Akt, cell proliferation, survival, migration and contraction, as well as PVR in a rabbit model. In addition, idelalisb prevented VEGF-induced cell proliferation, survival and tube formation, an in vitro angiogenesis model. Therefore, described herein are methods of using idelalisib to prevent (reduce the risk of) and treat PVR and intraocular pathological angiogenesis.

Subjects

The methods described herein can be used to prevent (reduce the risk of developing), or reduce the risk or rate of progression of abnormal intraocular diseases. For example, the methods can be used to prevent (reduce the risk of developing), or reduce the risk or rate of progression of PVR in patients, e.g., in patients requiring pars plana vitrectomy (PPV), e.g., for rhegmatagenous retinal detachment secondary to trauma; for patients requiring PPV for preexisting PVR grade C or higher; and/or for patients with retinal detachments requiring PPV for other indications associated with high risk condition for PVR development, e.g., giant retinal tears (giant retinal tears are defined as tears involving 90° or more of the circumference of the globe), retinal breaks larger than 3 disc areas, long-standing retinal detachments, or detachments associated with hemorrhage. In addition, the methods can be used to reduce the risk of developing, or the risk or rate of progression of, proliferative diabetic retinopathy (PDR), retinopathy of prematurity (ROP), and wet age-related macular degeneration (AMD).

The methods described herein can include identifying and/or selecting a subject who is in need of treatment to prevent the development of PVR or intraocular pathological angiogenesis (e.g., PDR, ROP and/or wet AMD) (e.g., selecting the subject on the basis of the need of treatment). In some embodiments, the subject is selected because they are at risk for PVR as a result of a condition listed above, e.g., an increased risk of developing PVR as a result of a condition listed above).

Proliferative Vitreoretinopathy (PVR)

The presentation of PVR clinically encompasses a wide phenotype. PVR can vary from a mild cellular haze (Grade A) to thick, fibrous membranes that cause the characteristic stiffened funnel of the detached retina (Grade D). A number of grading systems are in use, see, e.g., Ryan, Retina, 5^{th} ed (Elsevier 2013); Retina Society Terminology Committee. The classification of retinal detachment with proliferative vitreoretinopathy. Ophthalmology 1983; 90:121-5 (1983); Machemer R, Aaberg T M, Freeman H M, et al. Am J Ophthalmol 112:159-65 (1991); Lean J, Irvine A, Stern W, et al. Classification of PVR used in the silicone study. The Silicone study group. Ophthalmology 1989; 96:765 771. In some embodiments the methods include identifying, selecting, and/or treating a subject who has or is at risk of developing PVR. In some embodiments, the methods include monitoring the subject for early signs of the development of PVR, and administering one or more doses of idelalisib as described herein. The methods can also be used to treat subjects without present signs of PVR but who are at risk for PVR.

Proliferative Diabetic Retinopathy (PDR)

PDR is a common complication of diabetes mellitus and the leading cause of new blindness in persons aged 25-74 years in the United States. Signs of diabetic retinopathy (DR) include microaneurysms and hemorrhages (dot and blot, or flame-shaped); retinal edema and hard exudates; cotton-wool spots; venous loops and venous beading; and intraretinal microvascular abnormalities in a subject with diabetes (e.g., diagnosed based upon glucose and hemoglobin A1c measurements). The presence of neovascularization is a hallmark of PDR; in addition, preretinal hemorrhages, hemorrhage into the vitreous, fibrovascular tissue proliferation; traction retinal detachments, and macular edema may be present in PDR. Diagnosis is typically made by fluorescein angiography, Optical coherence tomography (OCT), or B-scan ultrasonography.

In some embodiments the methods include identifying, selecting, and/or treating a subject with diabetes who has or is at risk of developing PDR. In some embodiments, the methods include monitoring the subject for early signs of the development of PDR or DR, and administering one or more doses of idelalisib as described herein. The methods can also be used to treat subjects without present signs of PDR but who are at risk for PDR.

Standard treatments can include, e.g., intravitreal administration of triamcinolone, bevacizumab, or ranibizumab; laser photocoagulation; vitrectomy; or cryotherapy.

Retinopathy of Prematurity (ROP)

ROP affects immature vasculature in the eyes of premature babies, and can be mild with no visual defects or aggressive with neovascularization that can progress to retinal detachment and blindness. In some embodiments the methods include identifying, selecting, and/or treating a pre-term infant who has or is at risk of developing ROP. In some embodiments, the methods include monitoring the subject for early signs of the development of ROP, and administering one or more doses of idelalisib as described herein. The methods can also be used to treat subjects (e.g., preterm infants born before 32 weeks' gestation) without present signs of ROP but who are at risk for ROP.

Wet Age-Related Macular Degeneration (AMD)

In early stages of AMD insoluble extracellular aggregates called drusen accumulate in the retina. Advanced AMD occurs as either dry (atrophic) or wet (neovascular) AMD. In the former, geographic atrophy results in RPE atrophy, degeneration of the outer retinal layer, and sclerosis of choriocapillaris. Wet AMD is characterized by the presence of choroidal neovascularization (CNV): abnormal and immature blood vessels grow from the choroidal vasculature, through breaks in Bruch's membrane, toward the outer retina; these blood vessels leak fluid below or within the retina (Yanai et al., Proc Natl Acad Sci USA. 2014 Jul. 1; 111(26): 9603-9608; Wang et al., Eye (Lond). 2011 February; 25(2): 127-139). The two forms of AMD can occur together. Neovascular AMD accounts for 10 to 20% of AMD cases and leads to sudden and severe loss of vision (Ferris et al., Arch Ophthalmol. 1984 November; 102(11):1640-2). Current standard of care for patients with CNV/wet AMD involves targeting the proangiogenic and permeability molecule vascular endothelial growth factor-A (VEGF). However, although anti-VEGF therapy blocks vascular permeability and angiogenesis, it does not lead to complete vascular regression (Gragoudas et al., N Engl J Med. 2004 Dec. 30; 351(27):2805-16; Yanai et al., Proc Natl Acad Sci USA. 2014 Jul. 1; 111(26): 9603-9608) and the treatment is not effective in all subjects (Takeda et al., Nature. 2009 Jul. 9; 460(7252): 225-230).

In some embodiments the methods include identifying, selecting, and/or treating a subject who has CNV or wet AMD. In some embodiments, the methods include monitoring the subject for early signs of the development of CNV or AMD (e.g., presence of drusen), and administering one or more doses of idelalisib as described herein. The methods can also be used to treat subjects without present signs of CNV or wet AMD but who are at risk for CNV or wet AMD. Diagnosis of AMD or CNV can be made using known methods, e.g., Amsler grid, fluorescein angiography or Optical coherence tomography (OCT).

Methods of Treating or Reducing Risk of PVR and Intraocular Neovascularization

The methods described herein include the use of idelalisib in subjects who are at risk of developing a first or recurring PVR or intraocular neovascularization, e.g., a subject who is undergoing RD surgery as described above, and in subjects who have PVR, PDR, ROP, or AMD, or who are at risk for developing PVR PDR, ROP, or AMD.

In some embodiments, the methods described herein include the use of idelalisib in subjects who have undergone, are undergoing, or will undergo a pars plana vitrectomy (PPV) or scleral buckle (SB). In some embodiments, the methods include performing a PPV or RD surgery. Methods for performing these surgeries are known in the art; for example, typically, PPV is performed under local or general anesthesia using three, 23 or 20 gauge sclerotomy ports. Intraoperative tissue staining, perfluorocarbons, cryopexy, endolaser, scleral buckling, and lensectomy can also be performed as needed. Standard tamponading agents can be used, e.g., silicone oil or gas.

The methods described herein include the use of an effective amount of idelalisib. An “effective amount” is an amount sufficient to effect beneficial or desired results, e.g., the desired therapeutic effect (e.g., a prophylactically effective amount that reduces the risk of developing PVR). An effective amount can be administered in one or more administrations, applications or dosages. A therapeutically effective amount of idelalisib can be, e.g., 5-10 μM in the vitreous. The compositions can be administered, e.g., once per month or more after first administration. The skilled artisan will appreciate that certain factors may influence the dosage and timing required to effectively treat a subject, including but not limited to the severity of the disease or disorder, previous treatments, the general health and/or age of the subject, and other diseases present.

In some embodiments, intravitreal idelalisib injections are performed aseptically after the topical application of anaesthesia and an antiseptic agent to the conjunctival sac. In some embodiments, each subject receives an intravitreal injection of idelalisib.

In some embodiments, subjects who have undergone PPV receive multiple intravitreal injections of idelalisib during their post-operative period. The first injection can be administered intraoperatively; subsequently, injections can be administered, e.g., monthly basis. In some embodiments, the methods include additional doses at monthly frequency thereafter for an additional one, two, three, four, five, six, seven, eight, nine, ten, 11, or 12 months thereafter.

In some embodiments, the subjects receive a sustained release implant, e.g., as described above, that will release idelalisib over time, e.g., over a week, two weeks, a month, two months, three months, six months, or a year. In some embodiments, the methods include administering subsequent implants to provide idelalisib administration for at least six months, one year, two years, or more.

In some embodiments, idelalisib is administered in combination with one or more additional treatments, e.g., pharmaceutical treatments such as e.g., anti-VEGF agents (e.g., neutralizing VEGF antibodies (ranibizumab & bevacizumab) or recombinant fusion protein with the partial extracellular domains of VEGFR1 and 2 (aflibercept)) or corticosteroids (e.g., triamcinolone), or surgical treatments such as laser surgery (e.g., xenon, argon, diode), cryotherapy, pars plana vitrectomy (PPV), Retinal Detachment (RD) surgery; ERM surgery, scleral buckle surgery and/or vitrectomy.

Pharmaceutical Compositions and Methods of Administration

The methods described herein include the use of pharmaceutical compositions comprising idelalisib as an active ingredient. Thus also described herein are pharmaceutical compositions comprising idelalisib formulated for intravitreal or intraocular delivery.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., anti-VEGF agents (e.g., neutralizing VEGF antibodies (ranibizumab & bevacizumab) or recombinant fusion protein with the partial extracellular domains of VEGFR1 and 2 (aflibercept)) or corticosteroids (e.g., triamcinolone). Pharmaceutical compositions are typically formulated to be compatible with its intended route of administration. Examples of routes of administration suitable for use in the present methods can include intravitreal or intraocular administration, topical administration (e.g., eye drops), and intraocular implants. Systemic administration, e.g., oral administration can also be used.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). See also Short, Toxicol Pathol January 2008 vol. 36 no. 1 49-62. For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for intraocular or intravitreal injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared, e.g., by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. Nanoparticles (1 to 1,000 nm) and microparticles (1 to 1,000 pin), e.g., nanospheres and microspheres and nanocapsules and microcapsules, can also be used. These can be prepared according to methods known to those skilled in the art, for example, as described in U.S. Pat. No. 4,522,811; Bourges et al., Ocular drug delivery targeting the retina and retinal pigment epithelium using polylactide nanoparticles. Invest Opth Vis Sci 44:3562-9 (2003); Bourges et al., Intraocular implants for extended drug delivery: therapeutic applications. Adv Drug Deliv Rev 58:1182-1202 (2006); Ghate et al., Ocular drug delivery. Expert Opin Drug Deliv 3:275-87 (2006); and Short, Safety Evaluation of Ocular Drug Delivery Formulations: Techniques and Practical Considerations. Toxicol Pathol 36(1):49-62 (2008).

The pharmaceutical compositions can be included in a container, pack, or dispenser together with instructions for administration in a method described herein.

### EXAMPLES

The invention is further described in the following examples, which do not limit the scope of the invention described in the claims.

Material and Methods

The following Materials and Methods were used in the Examples below.

Major Reagents and Cell Culture

Primary antibodies against PDGFRα, p-PDGFRβ (p-Y751), PDGFR(3, p-Akt (p-S473), Akt, p-Erk, Erk, and p110δ were purchased from Cell Signaling Technology (Danvers, Mass.) and the β-Actin antibody was from Santa Cruz Biotechnology (Santa Cruz, Calif.). The anti-phospho-PDGFRα (Y742) antibody was produced as described previously^{44, 45}. HRP (horseradish peroxidase)-conjugated goat anti-rabbit IgG and goat anti-mouse IgG secondary antibodies were purchased from Santa Cruz Biotechnology. Enhanced chemiluminescent substrate for detection of horseradish peroxidase was from Pierce Protein Research Products (Rockford, Ill.). Idelalisib (a specific inhibitor for PI3Kδ) and GSK2636771 (a specific inhibitor for PI3Kβ) were purchased from APE×BIO (Houston, Tex.) and Cayman (Ann Arbor, Mich.), respectively. N-acetyl-cysteine and SU6656 (an inhibitor of Src family kinases) were purchased from Sigma (St. Luis, Mo.) and Calbiochem (San Diego, Calif.), respectively.

RPEM cells were RPE cells that were originated from an epiretinal membrane of a PVR patient as described previously²³. These RPEM cells, human primary RPE cells (hPRPE) (Lonza, Walkersville, Md.), ARPE-19 cells (American Type Culture Collection (ATCC), Manassas, Va.) and PAEC (porcine aortic endothelial cells)⁴⁶ were cultured in Dulbecco's modified Eagle's medium/nutrient mixture (DMEM/F12, Invitrogen, Grand Island, N.Y.) supplemented with 10% fetal bovine serum (FBS). Human corneal fibroblasts (HCF) were a gift from Dr. James D. Zieske lab⁴⁷ at Schepens Eye Research Institute (Boston, Mass.). Mouse cone photoreceptor cells (661W) were obtained by material transfer agreement from Department of Biomedical Engineering, University of Houston (Houston, Tex.)⁴⁸, respectively. HTB-18 (human retinoblastoma, ATCC), and RCF (rabbit conjuctival fibroblast) were obtained as described previously^{24, 49}). HREC: human retinal microvascular endothelial cells were from Cell Systems (Kirkland, Wash.), HUVEC: human umbilical vascular endothelial cells were from Lonza, and HLEC: human lymphatic endothelial cells were from ScienceCell (Carlsbad, Calif.). HCF, RCF, HTB-18, and 661W cells were cultured in DMEM supplemented with 10% FBS.

HEK 293T cells (HEK 293 containing SV40 T-antigen) from the Dana-Farber Cancer Institute/Harvard Medical School (Boston, Mass.) were cultured in high-glucose (4.5 g/L) DMEM supplemented with 10% FBS. All cells were cultured at 37° C. in a humidified 5% CO₂ atmosphere⁵⁰.

Construction of sgRNAs

To produce single guide RNA (sgRNA) for Streptococcus pyogenes (Sp) Cas (CRISPR-associated nucleases) 9 targets, we applied the CRISPR (clustered regularly interspaced short palindromic repeats) design tool (http://crispr.mit.edu) to select the four 20 nt target sequences preceding a 5P-NGG PAM (prospacer adjacent motif) sequence⁵¹ at the exon 3 in the human PDGFRB genomic locus (NG 023367.1). The four target sequences were target 1 (ACCCGAGCAGGTCAGAACGA (SEQ ID NO:8)), target 2 (CGGGTTCAGCTCCGGTGGTG (SEQ ID NO:9)), target 3 (GCCTGGTCGTCACACCCCCG (SEQ NO:10)) and target 4 (GCCCCGGGGGTGTGACGACC (SEQ ID NO:11)). Control sgRNA sequence (TGCGAATACGCCCACGCGATGGG (SEQ ID NO:12)) was to target the lacZ gene from Escherichia colI⁵¹.

In synthesizing sgRNAs, the oligos of top oligos 5′CACCG-20 targeted nucleotides and bottom oligos 5′-AAAC-20 nucleotides, which were complimentary to the target sequences, were synthesized, annealed and cloned into the lentiCRISPR v2 vector (Catalog number 52961, Addgene, Cambridge, Mass.) by BsmBl. All clones were confirmed by DNA sequencing using a primer 5′-GGACTATCATATGCTTACCG-3′ (SEQ ID NO:13) from the sequence of U6 promoter, which drove expression of sgRNAs.

Both synthesis of primers and oligos and sequencing of PCR products and clones were done by Massachusetts General Hospital (MGH) DNA Core Facility (Cambridge, Mass.).

Production of Lentivirus

The lentiCRISPR v2 vector inserted with sgRNA (2000 ng), the packaging plasmid pspax2 (Addgene: 12260) (900 ng) and the envelope plasmid VSV-G (Addgene: 8454) (100 ng) were mixed together and then added to a mixture of lipofectamine 3000 (Thermo Scientific) 6 μl with OPTI-MEM (Thermo Scientific, Waltham, Mass.) 90 μl. This transfection mix was incubated at room temperature for 30 minutes and then carefully transferred into a 60-mm cell culture dish with human embryonic kidney (HEK) 293T cells that were approximately 70% confluent without antibiotics. After 18 hours (37° C., 5% CO₂), the medium was replaced with growth medium supplemented with 30% FBS, and at 40 hours after the transfection, lentiviruses were harvested. The viral harvest was repeated at 24-hour intervals three times. The virus-containing media were pooled, centrifuged at 800×g for 5 minutes, and the supernatant was used to infect ARPE-19 and RPEM cells supplemented with 8 μg/ml polybrene (Sigma). The infected cells were selected in media with puromycin (4 μg/ml) and the resulting cells were examined by western blot^{24, 44, 50.}

Generation of RPEMβΔx

Construction of PDGFRβΔx was completed in two steps. First, the human PDGFRβΔx was cloned into the PVZ-ApaI-NotI-EcoRI-XbaI-SalI-PstI-HindII vector⁵² by EcoRI/XbaI. Then the PDGFRβΔx insert was subcloned as an EcoRI/SalI fragment into pLXSHD-EcoRI-HpaI-XhoI-BamHI vector by EcoRI/XhoI. The resultant construct was termed pLXSHD-PDGFRβΔK and verified by nucleotide sequencing at the MGH DNA core facility.

To make the retrovirus, the pLXSHD-PDGFRβΔx construct was transfected into 293GPG cells with reagent (Lipofectamine 2000; Invitrogen). Virus containing medium was collected for 5 days and then was concentrated (25,000g, 90 minutes, 4° C.). RPEM cells deficient for PDGFRβ were infected by incubation with the concentrated retrovirus in DMEM supplemented with 10% FBS and 8 μg/mL polybrene (hexadimethrine bromide; Sigma) for 24 hours. Successfully infected cells were selected in histidine-free DMEM supplemented with 2 mM L-histidinol dihydrochoride (Sigma, St. Louis, Mo.). Resultant cell lines were termed RPEMPAx; the level of the truncated PDGFRPAx was determined by Western blot analysis with an antiPDGFRβ antibody that recognizes the receptor's intracellular domain.

Western Blot

Cells grown to 90% confluence in wells of 24-well plates were serum starved for 24 hours, and then treated with appropriated agents. After two washes with ice-cold phosphate buffered saline (PBS), the cells were lysed in 1× sample buffer diluted with protein extraction buffer (10 mM Tris-HCl, pH 7.4; 5 mM ethylenediamineteraacetic acid (EDTA), 50 mM NaCl, 50 mM NaF, 1% Triton X-100, 20 μg/ml aprotinin, 2 mM Na₃VO₄, 1 mM phenylmethylsulfonyl fluoride] from 5× sample buffer [25 mM EDTA, 10% sodium dodecyl sulfate (SDS), 500 mM dithiothreitol (DTT), 50% sucrose, 500 mM Tris.HCl (pH=6.8), 0.5% bromophenol blue). The samples were boiled for 5 minutes and then centrifuged for 5 minutes at 13,000×g, 4° C. Proteins in the samples were separated by 10% SDS-polyacrylamide gel electrophoresis, transferred to polyvinylidene difluoride membranes, and then subjected to western blot analyses using appropriated antibodies. Signal intensity was determined by densitometry using NIH imageJ software^{24, 44, 50}.

Surveyor Nuclease Assay and DNA Sequencing.

The selected RPEM cells were detached and pelleted for genomic DNA extraction using the QuickExtract DNA Extraction Solution (Epicenter) by following the manufacturer's protocol. In brief, the pelleted cells were re-suspended in the QuickExtract solution, vortex for 15 seconds, at 65° C. for 6 min, vortex for 15 seconds and then at 98° C. for 5 min. The genomic region about 200 bp around the target 3 sequence was PCR amplified with high-fidelity Herculase II DNA polymerases (Agilent Technologies, Santa Clara, Calif.). The PCR primers (forward 5′-GGCGAGCTGCTGTTGCTGTC-3′ (SEQ ID NO:14) and reverse 5′-AGGTGCCATCCTGOCICCTIG-3′ (SEQ ID NO:15)) were synthesized by the MGH DNA core facility. The PCR products were separated in 2% agarose gel and purified with a gel extraction kit (Thermo Scientific, Waltham, Mass.) for Sanger DNA sequencing and next generation sequencing (NGS) by the MGH DNA Core facility and a Surveyor nuclease assay, which were performed according to the manufacturer's instructions (Integrated DNA Technologies, Coralville, Lowa). Briefly, the purified PCR products (300 ng) from the agarose gel were incubated with the surveyor nuclease and surveyor enhancer S with additional 1/10 MgCl2 (0.15 M) for 30 min at 42° C. and then separated by electrophoresis in a 2% agraose gel⁵³.

Cell Proliferation Assay

RPEM cells were seeded into wells of a 24-well plate at a density of 3×10⁴ cells/well in DMEM/F12 with 10% FBS. Following attachment, the cells were treated with DMEM/F12 or RV (1:2 dilution in DMEM/F12) with or without idelalisib (5 μM). On day 3, the cells were trypsin detached from the plates and counted in a hemocytometer. Each experimental condition was assayed in duplicate, and at least three independent experiments were performed^{24, 44}.

Cell Apoptosis Assay

RPEM cells were seeded into 6 cm-dishes at a density of 2×10⁵ cells per dish in DMEM/F12+10% FBS. Following attachment, the cells were treated with DMEM/F12 or RV (1:2 dilution in DMEM/F12) with or without idelalisib (504). On day 3, the cells were stained with fluorescein isothiocyanate (FITC)-conjugated annexin V and propidium iodide (PI) following the manufacturer's instructions (BD Biosciences, Palo Alto, Calif.). The cells were analyzed by flow cytometry in a Coulter Beckman XL instrument. At least three independent experiments were performed^{24, 44}.

Cell Migration Assay

RPEM cells in a 12-well plate were grown to near confluence, and then the wells with 90% confluent cells were scratched with 200 μl pipet tip. The cells were washed with PBS and treated with DMEM/F12 or RV (1:2 dilution in DMEM/F12) with or without idelalisib (5 μM). The scratched area was photographed to capture the initial width and photographed again 16 hours later. Analysis was conducted using Adobe Photoshop CS4 software. At least three independent experiments were performed.

Contraction Assay

RPEM cells were re-suspended in 1.5 mg/ml of neutralized collagen I (INAMED, Fremont, Calif.) (pH 7.2) at ice at a density 1×10⁶ cells/ml^{24, 54}. The mixture was transferred into wells of 24-well plates that had been preincubated overnight with 5 mg/ml bovine serum albumin/PBS. After the collagen had polymerized at 37° C. for 90 minutes, 0.5 ml DMEM/F12 or RV (1:2 dilution in DMEM/F12) with or without idelalisib (5 μM) was added. On day 3, the gel diameter was measured and the gel area was calculated using a formula 3.14×r², where r is the radius of the gel. At least three independent experiments were performed^{24, 55}.

Experimental PVR in Rabbits

As previously described^{1, 55}, PVR was induced in right eyes of two-three months old Dutch Belted rabbits purchased from Covance (Denver, Pa.). Briefly, a gas vitrectomy was performed by injecting 0.1 ml of perfluoropropane (C₃F₈) (Alcon, Fort Worth, Tex.) into the vitreous cavity 4 mm posterior to the corneal limbus. One week later, all rabbits were injected with platelet-rich plasma (0.1 ml) and 3.0×10⁵ cells of RPEM cells with idelalisib (final 10 μM) or its vehicle DMSO (final 0.01%) under an operative microscope. The retinal status was examined with an indirect ophthalmoscope plus a +30 D fundus lens on days 1, 3, 5, 7, 14, 21 and 28 by two double-masked ophthalmologists. PVR was graded according to the Fastenberg classification from 0 through 5⁵⁶. On day 28, animals were sacrificed, the eyes were enucleated, and the eyeballs were either fixed at 10% formalin for histology analysis or frozen at 80° C. for vitreous extraction. All surgeries were performed under aseptic conditions and adhered to the ARVO Statement for the Use of Animals in Ophthalmic and Vision Research. The protocol for the use of animals was approved by the Schepens Animal Care and Use Committee (Boston, Mass.). Rabbit vitreous (RV) was prepared from frozen rabbit eyeballs as described previously⁵⁰.

Electroretinogram (ERG) and Optical coherence Tomography (OCT)

On day 28, representative rabbits were in dark condition for one hour, and then the rabbits were deeply anesthetized with intramuscular anesthesia consisting of ketamine 30-50 mg/kg of body weight, xylazine 5-10 mg/kg of body weight and acepromazine 1 mg/kg of body weight. Depth of anesthesia was verified by the absence of the toe pinch withdrawal reflex. The pupils were dilated with topical 1% tropicamide to view the fundus⁵⁷. ERG analysis was performed as previously described⁵⁰. After ERG, fundus photographs and OCT were taken using a spectral domain (SD)-OCT system (Bioptigen Inc., Durham, N.C.).

### A Mouse Model of Oxygen-Induced Retinopathy

C57BL/6J litters on postnatal day (P) 7 were exposed to 75% oxygen until P12 in the oxygen chamber (Biospherix). Oxygen concentration was monitored daily using an oxygen sensor (Advanced Instruments, GPR-20F)^{58, 59}. On P12, the pups were anesthetized by intraperitoneal injection of 50 mg/kg ketamine hydrochloride and 10 mg/kg xylazine. During intravitreal injections, eyelids of P12 pups were separated by incision. Pupils were dilated using a drop of 1% tropicamide and the eyes were treated with topical proparacaine anesthesia. Intravitreous injections were performed under a microsurgical microscope using glass pipettes with a diameter of approximately 150 μm at the tip after the eye were punctured at the upper nasal limbus using a BD insulin syringe with the BD ultra-fine needle. One μl of idelalisib or DMSO was injected. After the intravitreal injection, the eyes were treated with a triple antibiotic (Neo/Poly/Bac) ointment and kept in room air (21% oxygen). On P17, the mice were euthanized and retinas were carefully removed and fixed in 3.7% paraformaldehyde (PFA), and the mice under 6 g were excluded from the experiments. In total there were six experiments performed in this OIR model. Retinal whole mounts were stained overnight at 4° C. with a murine-specific EC marker isolectin 4 (IB4)-Alexa 594^{46, 59, 69}. The images were taken with an EVOS FL Auto microscope (Life Technologies).

Statistics

The data were analyzed using an unpaired t test or a Mann Whitney test. A power (p) value less than 0.05 was considered statistically significant.

### Example 1. Idelalisib Inhibits Vitreous-Induced Activation of Akt and Pathogenesis of Retinal Pigment Epithelial Cells

Phosphoinositide (PI) 3-kinases (PI3Ks), a family of lipid kinases, phosphorylates the 3-hydroxyl of the inositol ring of inositol lipids for generation of PI(3)P, PI(3,4)P2, and PI(3,4,5)P3 at the inner leaflet of the plasma membrane³¹. Among the three main classes of PI3Ks, class I enzymes are receptor-regulated lipid kinases including heterodimeric PI3Kα, β, and δ³¹. Their regulatory subunit p85 that contains Src homology (SH)2 and SH3 domains can bind to the phosphorylated tyrosine in the Y-X-X-M motif of receptor tyrosine kinases, so that their catalytic subunit (p110α, β or δ) can phosphorylate PI(4,5)P2 to become PI(3, 4, 5)P₃, which in turn can be bound by pleckstrin homology (PH) domain-containing proteins such as Akt, an oncogene product also known as protein kinase B (Vanhaesebroeck et al., Nature reviews. Molecular cell biology 13, 195-203 (2012); Songyang et al., Cell 72, 767-778 (1993)). This binding facilitates Akt to be phosphorylated by PI-dependent kinase I at threonine 308 and by the mammalian target of rapamycin complex 2 at serine 473 (Sarbassov et al., Science 307, 1098-1101 (2005)). Activation of Akt can stimulate multiple cellular processes such as cell survival, proliferation, growth, as well as migration. So PI3Ks play an essential role in transmitting signals from cell surface molecules into the intracellular enzymes and in stimulating cellular responses (Vanhaesebroeck et al., Nature reviews. Molecular cell biology 13, 195-203 (2012); Somoza et al., J Biol Chem 290, 8439-8446 (2015)). As noted above, deregulation of PI3Ks may initiate multiple diseases such as cancer, angiogenesis and proliferative vitreoretinopathy (PVR) (Ikuno et al., Invest Ophthalmol Vis Sci 43, 483-489 (2002)).

PI3Kα and β are ubiquitously expressed and knockout of their subunit p110α or β is embryonic lethal, while mice without a catalytic subunit (p110δ) of PI3Kδ are viable (Somoza et al., J Biol Chem 290, 8439-8446 (2015); Vanhaesebroeck et al., Annual review of biochemistry 70, 535-602 (2001)). In humans, the highest levels of p1108 expression are seen in spleen and thymus (Chantry et al., J Biol Chem 272, 19236-19241 (1997)). However, the expressional pattern and function of p110δ in eye tissues have not been explored.

As shown in FIG. 1, p110δ was surprisingly highly expressed in all three RPE cell lines of ARPE-19 (a spontaneously arising RPE cell line), hPRPE (human primary RPE), and RPEM that were originated from an epiretinal membrane from a patient with PVR (FIG. 1A) (Wong et al., Can J Ophthalmol 37, 211-220 (2002)), whereas p110δ was very low or undetectable in other eye-originated cell lines examined including human corneal fibroblast, rabbit conjuctival fibroblast, human retinoblastoma cells and mouse 661W cone cells (FIG. 1A), demonstrating PI3Kδ was primarily expressed in RPE cells within the tested cell lines from the eye organ.

RPE has several functions (light absorption, epithelial transport, spartial ion buffering, visual cycle, phagocytosis, secretion and immune modulation) (Strauss, Physiological reviews 85, 845-881 (2005)) and is involved in age-related macular degeneration, retinitis pigmentosa, diabetic retinopathy and PVR (Andrews et al., Invest Ophthalmol Vis Sci 40, 2683-2689 (1999)). PI3Ks play an important role in experimental PVR (Ikuno et al., Invest Ophthalmol Vis Sci 43, 483-489 (2002); Lei et al., Mol Cell Biol 31, 1788-1799 (2011)), but which isoform of PI3Ks involving in this pathogenesis remained unknown. Thus, we examined whether idelalisib, a specific inhibitor of PI3Kδ, could prevent vitreous-induced activation of Akt in RPEM cells. Intriguingly, idelalisib at 1.0 μM completely inhibited vitreous-stimulated phosphorylation of Akt at serine 473 (FIG. 1B), while at its selective dose (10.0 μM) for PI3Kδ idelalisib neither inhibited vitreous-induced activation of Erk (FIG. 1B), nor PDGF-stimulated activation of Akt (FIG. 1B), indicating that idelalisib selectively blocks vitreous-induced activation of PI3Kδ/Akt pathway and that PDGF stimulates a different isoform of PI3Ks downstream of PDGFRs from the vitreous (FIGS. 5A-B) because in the vitreous we used in this research PDGF is undetectable by enzyme-linked immunosorbent assay (Lei et al., Invest Ophthalmol Vis Sci 50, 3394-3403 (2009)).

To demonstrate whether PDGFRβ played a role in vitreous-induced activation of Akt in the RPEM cells, we depleted expression of PDGFRβ in these cells using the technology of clustered regularly interspaced short palindromic repeats (CRISPR)-associated endonuclease (Cas)9 (FIGS. 6A-E and FIG. 2A). Subsequently, the RPEM cells with or without PDGFRβ were treated with vitreous for 30 minutes. Western blotting analysis showed that silence of PDGFRβ suppressed vitreous-stimulated activation of Akt (FIG. 2B). Next, we investigated how vitreous activated PDGFRβ/PI3Kδ/Akt since there was no PDGF in the vitreous we used. We hypothesized that vitreous activated this signaling pathway in RPEM cells similar to the activation of PDGFRα/PI3K/Akt in fibroblasts (Lei et al., J Biol Chem. 284, 6329-6336 (2009)). To test this hypothesis, we expressed a mutant PDGFRβ short of the PDGF-binding domain in the RPEM cells, whose wild type PDGFRβ had been silent by CRISPR/Cas9 editing (FIGS. 6A-E and FIG. 2B). As expected, expression of the mutant PDGFRβ restored vitreous-induced activation of Akt in the PDGFRβ silent cells (FIG. 2C); in addition, treatment with either a ROS scavenger N-acetyl-cysteine (NAC) or a Src-family kinase inhibitor (SU6656) prevented this restoration (FIG. 2B), indicating that both of ROS and Src activity are required for vitreous-induced activation of PDGFRP/PI3Kδ/Akt.

Activation of Akt can trigger a variety of cellular responses such as cell proliferation, survival and migration (Vanhaesebroeck et al., Nature reviews. Molecular cell biology 13, 195-203 (2012); Songyang et al., Cell 72, 767-778 (1993)). Thus, we investigated if idelalisib could inhibit vitreous-induced such cellular responses. As shown in FIG. 3, while vitreous stimulated proliferation, survival and migration of RPEM cells, idelalisib prevented these vitreous-induced cellular events. In the pathogenesis of PVR, membranes consisting of cells including RPE cells contract resulting in retinal detachment (Andrews et al., Invest Ophthalmol Vis Sci 40, 2683-2689 (1999)). To mimic this process, an in vitro collagen contraction assay was used to examine whether idelalisib could prevent vitreous-induced contraction of RPEM cells. As expected, vitreous stimulated the collagen contraction, and idelalisib completely blocked this event (FIG. 3D). These exciting results poised us to test whether idelalisib could prevent experimental PVR.

Next, the toxicity of idelalisib to cultured cells (FIG. 7) and rabbit eyes

(FIGS. 8A-C) was examined; the results indicated that intravitreally injected idelalisib at 10 μM was safe to rabbit eyes. Since idelalisib at 5 μM inhibited vitreous-induced signaling events and cellular responses in vitro, we investigated whether it at 10 μM could prevent experimental PVR in rabbits. As shown in FIG. 4, in the treatment with idelalisib, there was only one of nine rabbits developing to PVR stage 3, but all seven of eight control rabbits developed to severer PVR with retinal detachment (FIG. 4A). Importantly, idelalisib significantly suppressed the pathogenesis of PVR at the observed time points (days 7, 14, 21, and 28). To further confirm the PVR stages, representative rabbits were examined by ERG, OCT (FIGS. 4B & C) before euthanasia and histological analysis after euthanasia (FIG. 4D). These results identify p110δ as a novel target, and idelalisib as a novel therapeutic intervention, in RPE-related pathologies including PVR.

### Example 2. Idelalisib Inhibits VEGF-Induced Cell Proliferation, Migration and Tube Formation

Since idelalisib was able to block VEGF-induced activation of Akt, which plays a critical role in angiogenesis, we investigated whether idelalisib could inhibit VEGF-induced cell cellular responses involved in angiogenesis.

In a cell proliferation assay, HRECs cells were seeded into 24-well plates at a density of 30,000 cells/well in an endothelial growth medium kit. After attaching the plates, the cells were starved for growth factors for 7 hours, and then treated with VEGF (20 ng/ml) or VEGF plus idelalisib (5 μM). After 48 hours, the cells were trypsin detached and then counted in a hemocytometer under a light microscope as shown in FIG. 10A from three independent experiments^{50, 61}.

A wound healing assay was performed as previously described⁶² with minor modifications. Once cells reached 90% confluence in 48-well plates, they were starved for growth factors for 7 hours. A wound was created by scraping the cell monolayer with a sterile pipette tip (200 μl). The cells were washed twice to remove detached cells and then treated with VEGF (20 ng/ml) or VEGF plus idelalisib (5 μM). The wound was photographed at 18 hours post wounding with an EVOS FL Auto microscope (Thermo Scientific) as shown in 8B from three independent experiments⁶¹.

A tube formation assay was performed as previously described⁶³. A collagen gel mixture was added to a 96-well plate (70 μl/well), which was then incubated for about 60 minutes at 37° C. to let the collagen gel polymerize. After polymerization, 4.0×10⁴ HRECs were seeded in each well with their cultured medium maintained at a 37° C. incubator. This day was considered day 1. On day 2, the medium was removed and 150 μl of the gel mixture was added to each well supplemented with VEGF, or VEGF plus idelalisib (5 μM). On days 3, three different fields per well were randomly chosen and photographed using the EVOS FL Auto microscope. The results in FIG. 10B are representative of three independent experiments^{61, 64, 65}. Each bar graph indicates mean±standard deviation of three independent experiments. “*” denotes significant difference between the two compared groups. P<0.05 using an unpaired t-test.

As shown in FIGS. 10A-C, VEGF induced cell proliferation, migration and tube formation of HRECs as predicted, and idelalisib completely blocked VEGF-stimulated these cellular events, suggesting that idelalisib is capable of preventing VEGF-induced intraocular pathological angiogenesis.

### Example 3. Idelalisib Inhibits Neovascularization

Idelalisib inhibited angiogenesis in a mouse model of oxygen-induced retinopathy (OIR). Litters of P12 mice that had been exposed to 75% oxygen for five days were injected intravitreally with idelalisib. As shown in FIGS. 11A-B, idelalisib abrogated angiogenesis in a mouse model of oxygen-induced retinopathy.

### REFERENCES

[1] Andrews A, Balciunaite E, Leong F L, Tallquist M, Soriano P, Refojo M, Kazlauskas A: Platelet-derived growth factor plays a key role in proliferative vitreoretinopathy. Invest Ophthalmol Vis Sci 1999, 40:2683-9.
[2] Abrams G W, Azen, S. P., McCuen, B. W., 2nd, Flynn, H. W., Jr., Lai, M. Y., and Ryan, S. J.: Vitrectomy with silicone oil or long-acting gas in eyes with severe proliferative vitreoretinopathy: results of additional and long-term follow-up. Silicone Study report 11. Arch Ophthalmol 1997, 115:335-44.
[3] Pastor-Idoate S, Rodriguez-Hernandez I, Rojas J, Fernandez I, Garcia-Gutierrez M T, Ruiz-Moreno J M, Rocha-Sousa A, Ramkissoon Y, Harsum S, MacLaren R E, Charteris D, VanMeurs J C, Gonzalez-Sarmiento R, Pastor J C, Genetics on PVRSG: The T309G MDM2 gene polymorphism is a novel risk factor for proliferative vitreoretinopathy. PloS one 2013, 8:e82283.
[4] Casaroli-Marano R P, Pagan R, Vilaro S: Epithelial-mesenchymal transition in proliferative vitreoretinopathy: intermediate filament protein expression in retinal pigment epithelial cells. Invest Ophthalmol Vis Sci 1999, 40:2062-72.
[5] Connor T B, Jr., Roberts A B, Sporn M B, Danielpour D, Dart L L, Michels R G, de Bustros S, Enger C, Kato H, Lansing M, et al.: Correlation of fibrosis and transforming growth factor-beta type 2 levels in the eye. J Clin Invest 1989, 83:1661-6.
[6] Leaver P K, Billington B M: Vitrectomy and fluid/silicone-oil exchange for giant retinal tears: 5 years follow-up. Graefes Arch Clin Exp Ophthalmol 1989, 227:323-7.
[7] Cui J, Lei H, Samad A, Basavanthappa S, Maberley D, Matsubara J, Kazlauskas A: PDGF receptors are activated in human epiretinal membranes. Exp Eye Res 2009, 88:438-44.
[8] Schmidt G W, Broman A T, Hindman H B, Grant M P: Vision survival after open globe injury predicted by classification and regression tree analysis. Ophthalmology 2008, 115:202-9.
[9] Agrawal R N, He S, Spee C, Cui J Z, Ryan S J, Hinton D R: In vivo models of proliferative vitreoretinopathy. Nat Protoc 2007, 2:67-77.
[10] Cleary P E, Ryan S J: Experimental posterior penetrating eye injury in the rabbit.
11. Histology of wound, vitreous, and retina. Br J Ophthalmol 1979, 63:312-21.
[11] Cleary P E, Ryan S J: Experimental posterior penetrating eye injury in the rabbit. I. Method of production and natural history. Br J Ophthalmol 1979, 63:306-11.
[12] Erdurman F C, Hurmeric V, Gokce G, Durukan A H, Sobaci G, Altinsoy H I: Ocular injuries from improvised explosive devices. Eye (Lond) 2011, 25:1491-8.
[13] Weichel E D, Colyer M H, Ludlow S E, Bower K S, Eiseman A S: Combat ocular trauma visual outcomes during operations iraqi and enduring freedom. Ophthalmology 2008, 115:2235-45.
[14] Zhou Q, Xu G, Zhang X, Cao C, Zhou Z: Proteomics of post-traumatic proliferative vitreoretinopathy in rabbit retina reveals alterations to a variety of functional proteins. Curr Eye Res 2012, 37:318-26.
[15] Vergara O, Ogden T, Ryan S: Posterior penetrating injury in the rabbit eye: effect of blood and ferrous ions. Exp Eye Res 1989, 49:1115-26.
[16] Negrel A D, Thylefors B: The global impact of eye injuries. Ophthalmic epidemiology 1998, 5:143-69.
[17] Stahl A, Connor K M, Sapieha P, Chen J, Dennison R J, Krah N M, Seaward M R, Willett K L, Aderman C M, Guerin K I, Hua J, Lofqvist C, Hellstrom A, Smith L E: The mouse retina as an angiogenesis model. Invest Ophthalmol Vis Sci 2010, 51:2813-26.
[18] Williams R, Airey M, Baxter H, Forrester J, Kennedy-Martin T, Girach A: Epidemiology of diabetic retinopathy and macular oedema: a systematic review. Eye (Lond) 2004, 18:963-83.
[19] Fraser-Bell S, Kaines A, Hykin P G: Update on treatments for diabetic macular edema. Current opinion in ophthalmology 2008, 19:185-9.
[20] Mintz-Hittner H A, Kennedy K A, Chuang A Z, Group B-R C: Efficacy of intravitreal bevacizumab for stage 3+retinopathy of prematurity. N Engl J Med 2011, 364:603-15.
[21] Chakravarthy U, Harding S P, Rogers C A, Downes S M, Lotery A J, Culliford L A, Reeves B C, investigators Is: Alternative treatments to inhibit VEGF in age-related choroidal neovascularisation: 2-year findings of the IVAN randomised controlled trial. Lancet 2013, 382:1258-67.
[22] Ying G S, Maguire M G, Daniel E, Ferris F L, Jaffe G J, Grunwald J E, Toth C A, Huang J, Martin D F, Comparison of Age-Related Macular Degeneration Treatments Trials Research G: Association of Baseline Characteristics and Early Vision Response with 2-Year Vision Outcomes in the Comparison of AMD Treatments Trials (CATT). Ophthalmology 2015, 122:2523-31 e1.
[23] Wong C A, Potter M J, Cui J Z, Chang T S, Ma P, Maberley A L, Ross W H, White V A, Samad A, Jia W, Hornan D, Matsubara J A: Induction of proliferative vitreoretinopathy by a unique line of human retinal pigment epithelial cells. Can J Ophthalmol 2002, 37:211-20.
[24] Lei H, Velez G, Cui J, Samad A, Maberley D, Matsubara J, Kazlauskas A: N-Acetylcysteine Suppresses Retinal Detachment in an Experimental Model of Proliferative Vitreoretinopathy. Am J Pathol 2010, 177:132-40.
[25] Somoza J R, Koditek D, Villasenor A G, Novikov N, Wong MEI, Liclican A, Xing W, Lagpacan L, Wang R, Schultz B E, Papalia G A, Samuel D, Lad L, McGrath M E: Structural, biochemical, and biophysical characterization of idelalisib binding to phosphoinositide 3-kinase delta. J Biol Chem 2015, 290:8439-46.
[26] Vanhaesebroeck B, Welham M J, Kotani K, Stein R, Warne P H, Zvelebil M J, Higashi K, Volinia S, Downward J, Waterfield M D: P110delta, a novel phosphoinositide 3-kinase in leukocytes. Proc Natl Acad Sci USA 1997, 94:4330-5.
[27] Ali K, Bilancio A, Thomas M, Pearce W, Gilfillan A M, Tkaczyk C, Kuehn N, Gray A, Giddings J, Peskett E, Fox R, Bruce I, Walker C, Sawyer C, Okkenhaug K, Finan P, Vanhaesebroeck B: Essential role for the p110delta phosphoinositide 3-kinase in the allergic response. Nature 2004, 431:1007-11.
[28] Songyang Z, Shoelson S E, Chaudhuri M, Gish G, Pawson T, Haser W G, King F, Roberts T, Ratnofsky S, Lechleider R J, et al.: SH2 domains recognize specific phosphopeptide sequences. Cell 1993, 72:767-78.
[29] Yoakim M, Hou W, Songyang Z, Liu Y, Cantley L, Schaffhausen B: Genetic analysis of a phosphatidylinositol 3-kinase SH2 domain reveals determinants of specificity. Mol Cell Biol 1994, 14:5929-38.
[30] Stephens L R, Eguinoa A, Erdjument-Bromage H, Lui M, Cooke F, Coadwell J, Smrcka A S, Thelen M, Cadwallader K, Tempst P, Hawkins P T: The G beta gamma sensitivity of a PI3K is dependent upon a tightly associated adaptor, p101. Cell 1997, 89:105-14.
[31] Vanhaesebroeck B, Stephens L, Hawkins P: PI3K signalling: the path to discovery and understanding. Nature reviews Molecular cell biology 2012, 13:195-203.
[32] Sarbassov D D, Guertin D A, Ali S M, Sabatini D M: Phosphorylation and regulation of Akt/PKB by the rictor-mTOR complex. Science 2005, 307:1098-101.
[33] Franke T F, Kaplan D R, Cantley L C, Toker A: Direct regulation of the Akt proto-oncogene product by phosphatidylinositol-3,4-bisphosphate. Science 1997, 275:665-8.
[34] Gerber H P, McMurtrey A, Kowalski J, Yan M, Keyt B A, Dixit V, Ferrara N: Vascular endothelial growth factor regulates endothelial cell survival through the phosphatidylinositol 3′-kinase/Akt signal transduction pathway. Requirement for Flk-1/KDR activation. J Biol Chem 1998, 273:30336-43.
[35] Kitamura T, Asai N, Enomoto A, Maeda K, Kato T, Ishida M, Jiang P, Watanabe T, Usukura J, Kondo T, Costantini F, Murohara T, Takahashi M: Regulation of VEGF-mediated angiogenesis by the Akt/PKB substrate Girdin. Nat Cell Biol 2008, 10:329-37.
[36] Luo J, Manning B D, Cantley L C: Targeting the PI3K-Akt pathway in human cancer: rationale and promise. Cancer Cell 2003, 4:257-62.
[37] Kureishi Y, Luo Z, Shiojima I, Bialik A, Fulton D, Lefer D J, Sessa W C, Walsh K: The HMG-CoA reductase inhibitor simvastatin activates the protein kinase Akt and promotes angiogenesis in normocholesterolemic animals. Nat Med 2000, 6:1004-10.
[38] Luo Z, Fujio Y, Kureishi Y, Rudic R D, Daumerie G, Fulton D, Sessa W C, Walsh K: Acute modulation of endothelial Akt/PKB activity alters nitric oxide-dependent vasomotor activity in vivo. J Clin Invest 2000, 106:493-9.
[39] Dimmeler S, Zeiher A M: Akt takes center stage in angiogenesis signaling. Circ Res 2000, 86:4-5.
[40] Ikuno Y, Leong F L, Kazlauskas A: Attenuation of experimental proliferative vitreoretinopathy by inhibiting the platelet-derived growth factor receptor. Invest Ophthalmol Vis Sci 2000, 41:3107-16.
[41] Ikuno Y, Kazlauskas A: An in vivo gene therapy approach for experimental proliferative vitreoretinopathy using the truncated platelet-derived growth factor alpha receptor. Invest Ophthalmol Vis Sci 2002, 43:2406-11.
[42] Ikuno Y, Leong F L, Kazlauskas A: PI3K and PLCgamma play a central role in experimental PVR. Invest Ophthalmol Vis Sci 2002, 43:483-9.
[43] Lei H, Velez G, Kazlauskas A: Pathological signaling via platelet-derived growth factor receptor {alpha} involves chronic activation of Akt and suppression of p53. Mol Cell Biol 2011, 31:1788-99.
[44] Lei H, Kazlauskas A: Growth factors outside of the PDGF family employ ROS/SFKs to activate PDGF receptor alpha and thereby promote proliferation and survival of cells. J Biol Chem 2009, 284:6329-36.
[45] Lei H, Kazlauskas A: A reactive oxygen species-mediated, self-perpetuating loop persistently activates platelet-derived growth factor receptor alpha. Mol Cell Biol 2014, 34:110-22.
[46] Lei H, Romeo G, Kazlauskas A: Heat shock protein 90alpha-dependent translocation of annexin II to the surface of endothelial cells modulates plasmin activity in the diabetic rat aorta. Circ Res 2004, 94:902-9.
[47] Guo X, Hutcheon A E, Melotti S A, Zieske J D, Trinkaus-Randall V, Ruberti J W: Morphologic characterization of organized extracellular matrix deposition by ascorbic acid-stimulated human corneal fibroblasts. Invest Ophthalmol Vis Sci 2007, 48:4050-60.
[48] Tan E, Ding X Q, Saadi A, Agarwal N, Naash M I, Al-Ubaidi M R: Expression of cone-photoreceptor-specific antigens in a cell line derived from retinal tumors in transgenic mice. Invest Ophthalmol Vis Sci 2004, 45:764-8.
[49] Lei H, Rheaume M A, Cui J, Mukai S, Maberley D, Samad A, Matsubara J, Kazlauskas A: A novel function of p53: a gatekeeper of retinal detachment. Am J Pathol 2012, 181:866-74.
[50] Lei H, Qian C X, Lei J, Haddock L J, Mukai S, Kazlauskas A: RasGAP Promotes Autophagy and Thereby Suppresses Platelet-Derived Growth Factor Receptor-Mediated Signaling Events, Cellular Responses, and Pathology. Mol Cell Biol 2015, 35:1673-85.
[51] Swiech L, Heidenreich M, Banerjee A, Habib N, Li Y, Trombetta J, Sur M, Zhang F: In vivo interrogation of gene function in the mammalian brain using CRISPR-Cas9. Nature biotechnology 2015, 33:102-6.
[52] Drummond-Barbosa D A, Vaillancourt R R, Kazlauskas A, DiMaio D: Ligand-independent activation of the platelet-derived growth factor beta receptor: requirements for bovine papillomavirus E S-induced mitogenic signaling. Mol Cell Biol 1995, 15:2570-81.
[53] Ran F A, Hsu P D, Lin C Y, Gootenberg J S, Konermann S, Trevino A E, Scott D A, Inoue A, Matoba S, Zhang Y, Zhang F: Double nicking by RNA-guided CRISPR Cas9 for enhanced genome editing specificity. Cell 2013, 154:1380-9.
[54] Lei H, Velez G, Hovland P, Hirose T, Gilbertson D, Kazlauskas A: Growth factors outside the PDGF family drive experimental PVR. Invest Ophthalmol Vis Sci 2009, 50:3394-403.
[55] Lei H, Rheaume M A, Velez G, Mukai S, Kazlauskas A: Expression of PDGFR{alpha} Is a Determinant of the PVR Potential of ARPE19 Cells. Invest Ophthalmol Vis Sci 2011, 52:5016-21.
[56] Fastenberg D M, Diddie K R, Sorgente N, Ryan S J: A comparison of different cellular inocula in an experimental model of massive periretinal proliferation. Am J Ophthalmol 1982, 93:559-64.
[57] Giani A, Luiselli C, Esmaili D D, Salvetti P, Cigada M, Miller J W, Staurenghi G: Spectral-domain optical coherence tomography as an indicator of fluorescein angiography leakage from choroidal neovascularization. Invest Ophthalmol Vis Sci 2011, 52:5579-86.
[58] Saint-Geniez M, Jiang A, Abend S, Liu L, Sweigard H, Connor K M, Arany Z: PGC-lalpha regulates normal and pathological angiogenesis in the retina. Am J Pathol 2013, 182:255-65.
[59] Connor K M, Krah N M, Dennison R J, Aderman C M, Chen J, Guerin K I, Sapieha P, Stahl A, Willett K L, Smith L E: Quantification of oxygen-induced retinopathy in the mouse: a model of vessel loss, vessel regrowth and pathological angiogenesis. Nat Protoc 2009, 4:1565-73.
[60] Kawamoto A, Gwon H C, Iwaguro H, Yamaguchi J I, Uchida S, Masuda H, Silver M, Ma H, Kearney M, Isner J M, Asahara T: Therapeutic potential of ex vivo expanded endothelial progenitor cells for myocardial ischemia. Circulation 2001, 103:634-7.
[61] Ruan G X, Kazlauskas A: Axl is essential for VEGF-A-dependent activation of PI3K/Akt. EMBO J 2012, 31:1692-703.
[62] Liang C C, Park A Y, Guan J L: In vitro scratch assay: a convenient and inexpensive method for analysis of cell migration in vitro. Nat Protoc 2007, 2:329-33.
[63] Im E, Venkatakrishnan A, Kazlauskas A: Cathepsin B regulates the intrinsic angiogenic threshold of endothelial cells. Molecular biology of the cell 2005, 16:3488-500.
[64] Arnaoutova I, Kleinman H K: In vitro angiogenesis: endothelial cell tube formation on gelled basement membrane extract. Nat Protoc 2010, 5:628-35.
[65] Im E, Kazlauskas A: Regulating angiogenesis at the level of PtdIns-4,5-P2. EMBO J 2006, 25:2075-82.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating or reducing the risk of intraocular diseases associated with angiogenesis, in a subject, the method comprising administering a therapeutically effective amount of idelalisib, preferably via intravitreal injection.

2. The method of claim 1, wherein the intraocular disease is proliferative vitreoretinopathy (PVR) or intraocular pathological neovascularization.

3. The method of claim 1, wherein the intraocular pathological neovascularization is proliferative diabetic retinopathy (PDR), retinopathy of prematurity (ROP), or wet age-related macular degeneration (AMD).

4. The method of claim 1, wherein the subject is undergoing or has undergone an ocular surgical procedure that increases the subject's risk of developing PVR; has diabetes; is a pre-term infant born before 32 weeks' gestation; or has early stage AMD.

5. The method of claim 4, wherein the subject has diabetic retinopathy.

6. The method of claim 4, wherein the ocular surgical procedure is a pars plana vitrectomy (PPV), Retinal Detachment (RD) surgery; ERM surgery; scleral buckle surgery; or a procedure in the other eye.

7. The method of claim 6, wherein the subject requires a PPV to treat a rhegmatagenous retinal detachment secondary to trauma; preexisting proliferative vitreoretinopathy; or for other indications associated with high risk condition for PVR development.

8. The method of claim 7, wherein the indication associated with high risk condition for PVR development is a giant retinal tear, a retinal break larger than 3 disc areas, a long-standing retinal detachment, or a detachment associated with hemorrhage.

9. A composition comprising idelalisib formulated for intravitreal injection.

10.-16. (canceled)
